(19) United States
(12) Patent Application Publication
(10) Pub. No.: US 2025/0380861 A1
(43) Pub. Date: Dec. 18, 2025

(54) BRUSH FOR ENDOSCOPE REPROCESSING

(71) Applicant: Boston Scientific Scimed, Inc., Maple Grove, MN (US)

(72) Inventors: José Fernando Gomez Bonilla, Bogotá (CO); Hugo Felipe Chica Cardona, Bogotá (CO); Juan Pablo Herrera Meza, Bogotá D.C (CO); Santiago Sanchez Renteria, Bogotá D.C (CO); Daniela Cespedes Valenzuela, Bogotá (CO); Carolina Villarreal, Hopedale, MA (US)

(73) Assignee: Boston Scientific Scimed, Inc., Maple Grove, MN (US)

(21) Appl. No.: 19/233,832

(22) Filed: Jun. 10, 2025

Related U.S. Application Data
(60) Provisional application No. 63/659,171, filed on Jun. 12, 2024.

(51) Int. Cl. A61B 1/12; A46B 5/00; A46B 5/06; A46B 9/02

(52) U.S. Cl. CPC A61B 1/122; A46B 5/0012; A46B 5/06; A46B 9/026; A46B 2200/3013

(57) ABSTRACT

Devices, systems, and methods for cleaning channels within an endoscopic device. A brush may be used for cleaning an endoscopic medical device, for example, for cleaning a channel of the endoscopic medical device. The brush may include a wire, a plurality of receiver bodies disposed along the wire and attached to the wire. Each receiver body may have a concave shape oriented in a same direction along the wire defining a front direction. In front of each of the plurality of receiver bodies there may be a plurality of bristles attached to the wire and extending transversely from the wire.

### CROSS-REFERENCE TO RELATED APPLICATIONS

This application claims the benefit of U.S. Provisional Patent Application Ser. No. 63/659,171 filed on Jun. 12, 2024, the disclosure of which is incorporated herein by reference.

### FIELD

This disclosure relates generally to cleaning devices and methods, and particularly for cleaning devices for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages.

Because of the high costs associated with manufacturing these components, many are designed to be reusable. However, the reuse of endoscopic medical devices is in tension with the need for a sterile medical environment. There is therefore a need for efficient and effective procedures to thoroughly clean and sanitize endoscopic devices prior to reuse.

### SUMMARY

This disclosure provides design, material, manufacturing method, and use alternatives for medical devices and medical systems. In a first example, a brush for cleaning an endoscopic medical device includes a wire; a plurality of receiver bodies disposed along the wire and attached to the wire, each receiver body having a concave shape oriented in the same direction along the wire defining a front direction; and, in front of each of the plurality of receiver bodies, a plurality of bristles extending transversely from the wire and attached to the wire.

Alternatively or additionally to any of the examples above, a rearmost body of the plurality of receiver bodies can be larger than a frontmost body of the plurality of receiver bodies.

Alternatively or additionally to any of the examples above, the plurality of receiver bodies can consist of a front body, a middle body, and a rear body.

Alternatively or additionally to any of the examples above, the front body can have a diameter smaller than a diameter of the middle body, and the diameter of the middle body can be smaller than a diameter of the rear body.

Alternatively or additionally to any of the examples above, each of the receiver bodies can be made of the same material. The material can be viscoelastic.

Alternatively or additionally to any of the examples above, the bristles can be oriented transverse from the wire and radially about the wire to form a cylindrical volume defining a diameter.

Alternatively or additionally to any of the examples above, a rearmost body of the plurality of receiver bodies can have a diameter that is larger than the diameter defined by the bristles.

Alternatively or additionally to any of the examples above, a frontmost body of the plurality of receiving bodies can have a receiver that is equal to the diameter defined by the bristles.

Alternatively or additionally to any of the examples above, the wire can extend to the front of the brush a length significantly longer than a total length of the wire including the receiver bodies and the bristles.

Alternatively or additionally to any of the examples above, the brush can further include a plurality of router bodies disposed along the wire, each router body in front of a different body of the plurality of receiver bodies

Alternatively or additionally to any of the examples above, each of the router bodies and each of the receiver bodies can be made of the same viscoelastic material.

In another example, a method of cleaning an endoscopic channel includes passing a brush of any of the examples above through the channel; and applying a fluid to rinse the endoscopic channel.

Alternatively or additionally to any of the examples above, the method can further include inspecting the channel for debris; and determining that the endoscopic channel is free of debris after a single pass of the brush through the channel.

Alternatively or additionally to any of the examples above, the method can further include subjecting the endoscopic channel to high level disinfection.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 is a flowchart illustrating an exemplary medical device cleaning process;

FIG. 4 depicts a schematic view of an illustrative brush;

FIGS. 5A-5D depict a schematic cross-section view of an illustrative brush used in cleaning an illustrative endoscopic channel; and

FIG. 6 depicts a schematic view of an illustrative brush.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

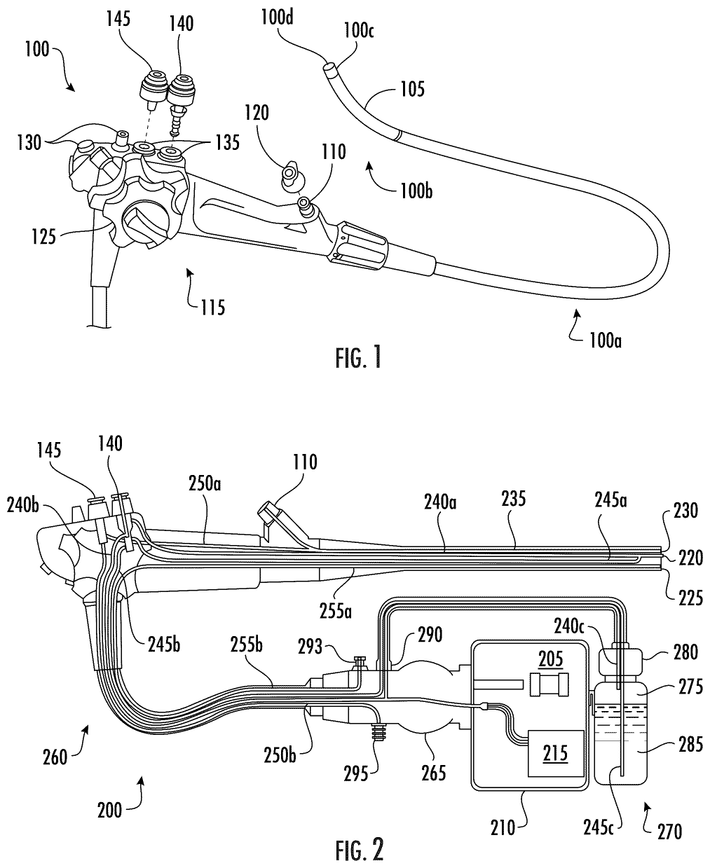

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the pressurizing pump 215 to the gas feed line 240b in the umbilical 260.

A water container or reservoir 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the pressurizing pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

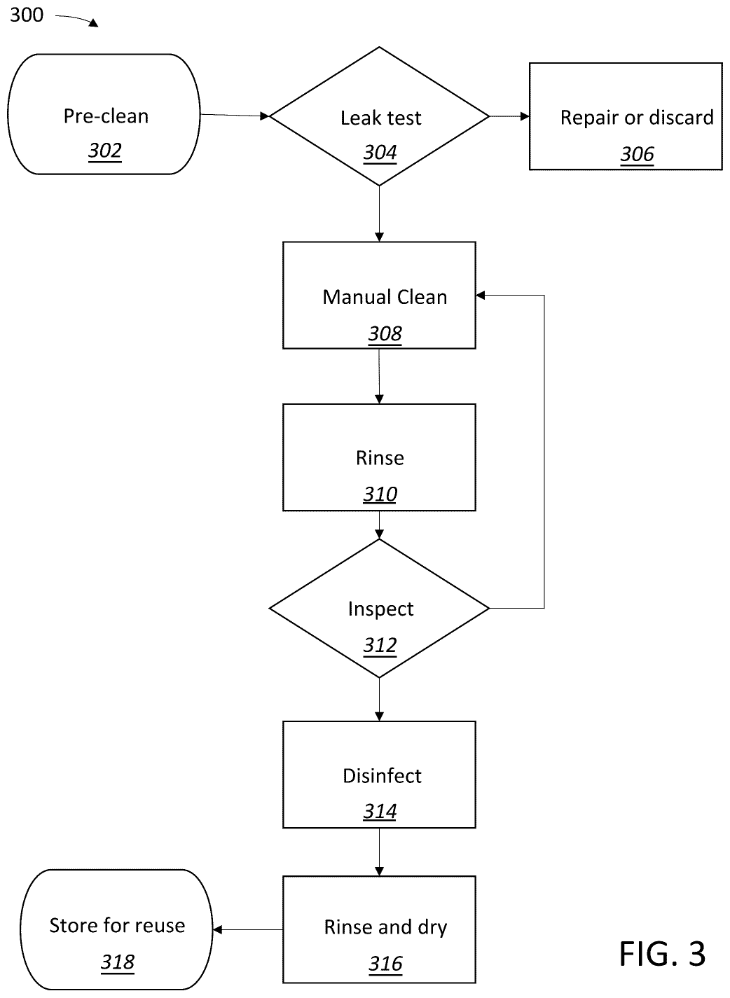

After the use of the above system 200 in an endoscopic procedure, some of the components may be reprocessed for subsequent reuse. An example of such a reprocessing method 300 is shown in FIG. 3.

In step 302, components of the system are subject to initial cleaning. This pre-cleaning step may be carried out immediately after the endoscopic procedure or at some later time. Pre-cleaning may involve an initial rinse and/or immersion to rid components of any visible external fluid or detritus. In some implementations, pre-cleaning may be performed at a cleaner's discretion based on informal or casual inspection of the components to be cleaned.

Following pre-cleaning, in step 304, leak testing may be performed on one or more of the components, particularly the tubing. This may involve running a sterile liquid, such as water or saline solution, through the supply and/or working channels to ensure that no liquid escapes. Should any component be found to have a leak, it may be set aside for disposal or repair (step 306), and only components that pass the leak testing complete the remaining steps of the reprocessing as described.

A manual cleaning is performed at step 308. Here, brushes and other tools are used to remove any debris. It is during this manual cleaning step that, in some implementations, a cleaning brush of the sort described herein may be used. The handle 115 and any of the tubing described herein may be cleaned. Particularly the working channel 235 and any other tubing that is exposed to biological material such as patient fluids, samples, or waste may be included in the manual cleaning. For each channel to be cleaned, cleaning tools such as the cleaning brush are inserted and pushed entirely through the channel to capture and remove the debris in that channel.

One or more rinsing cycles follow the manual cleaning at step 310, which may involve any appropriate cleaning liquid such as distilled water, saline, detergent solution, or the like. A visual inspection is then performed (step 312) to confirm the efficacy of the cleaning and rinsing steps; a failed inspection may then require more manual cleaning (returning to step 308).

Should the inspection confirm that the component or components are satisfactorily free of debris, the devices may then be disinfected at step 314. The disinfecting process may involve the exposure of components to radiation, temperature, liquids, gases, or any combination of these, and may be carried out over any time period sufficient to thoroughly clean the device to the standards necessary for medical reuse. In some implementations, the disinfection process may meet regulatory and industry standards for High-Level Disinfection. Following the disinfection process, the components are again rinsed and dried to remove any residue (step 316) prior to being stored for reuse (step 318).

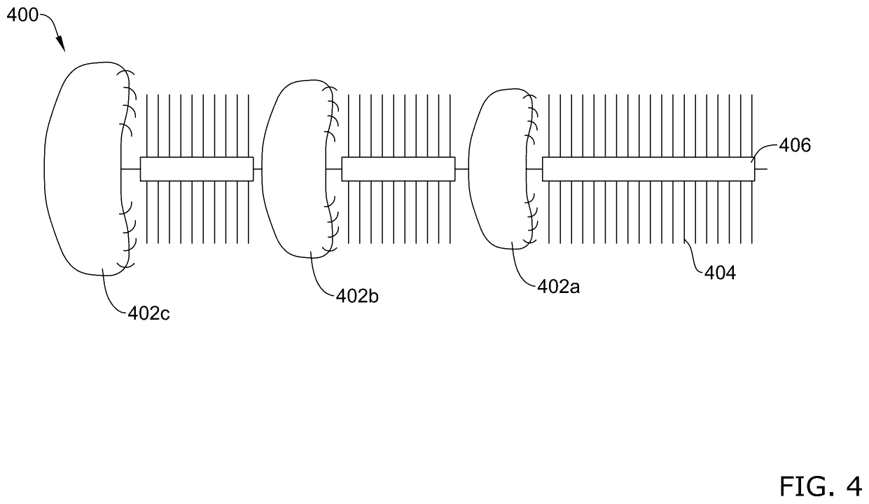

A brush 400 for use in the manual cleaning step is shown in FIG. 4. The brush 400 comprises three receiver bodies 402a, 402b, and 402c separated by bristles 404. Each of the receivers 402a-c is formed of a viscoelastic, non-absorbent material. The receivers 402a-c are each concave and are open toward the front of the brush 400, defined as the direction in which the brush will be pulled through the channel to be cleaned. In some implementations, the receiver bodies 402a, 402b, and 402c are each a different diameter, such that the rearmost receiver 402c is the largest and the frontmost receiver 402a is the smallest.

The receivers are spaced along a brush wire 406. Then wire 406 may be made of any material both flexible enough to follow the endoscopic channel and strong enough to support the components of the brush moving through the channel, such as metal, plastic, nylon, filament, or the like. In front of each of the receivers 402a-c, the brush wire 406 includes bristles 404. The bristles 404 are semi-flexible pieces of material appropriate for scrubbing the interior surface of the endoscopic channel. In some implementations, the bristles 404 may be a plastic, nylon, or metal substance of appropriate durometer. The bristles 404 may be generally non-absorbent and water resistant.

The brush wire 406 is flexible to allow movement of the brush 400 through the endoscopic channel to be cleaned. In some implementations, the brush wire 406 may include a significant length past the front receiver 402a used to pull the brush 400 through the endoscopic channel. In other implementations, a separate pull wire may be attached at the front of the brush 400 during the cleaning process.

The size of the components of the brush 400 may be suited for ideal use in a particular endoscopic channel. For example, the bristles 404 may be of a radius to slightly exceed the radius of the channel interior to ensure that they contact and scrub along the interior surface during cleaning. Each of the receiver bodies 402a-c may be larger in diameter than the interior surface such that each receiver body must deform (squeeze) to fit within the channel.

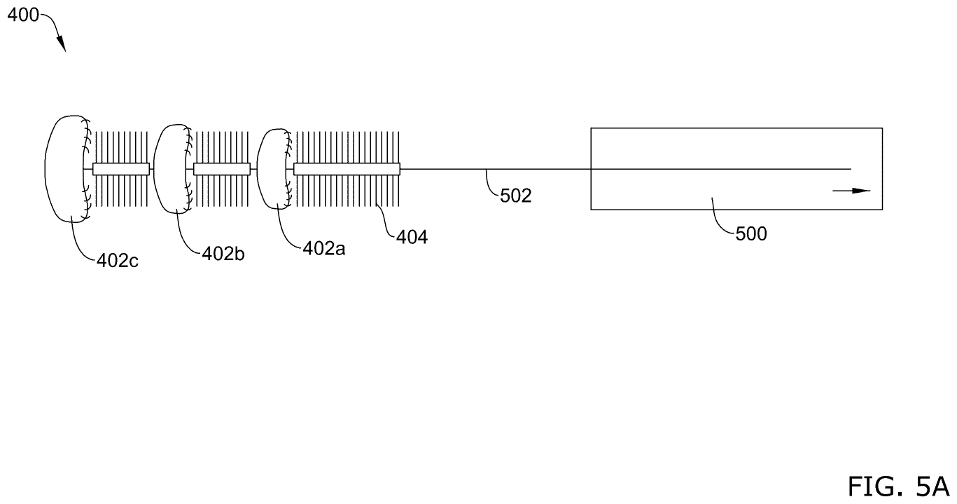

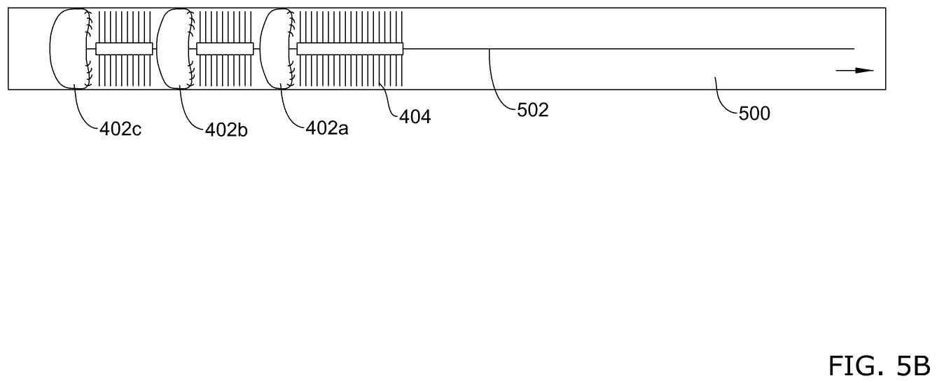

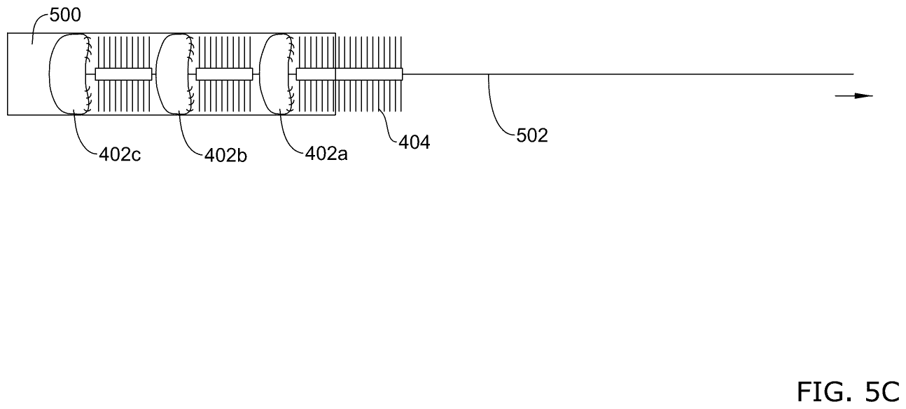

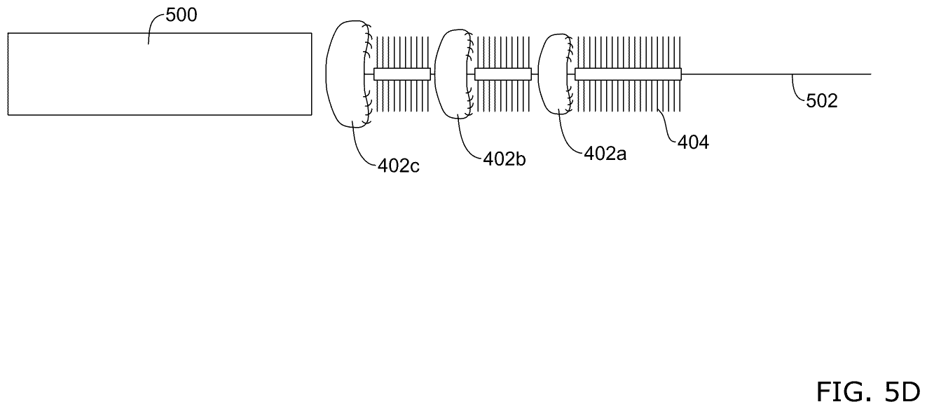

FIG. 5A-5D illustrate a channel 500 through which a brush 400 is moved. A pull wire 502, which may be integral with or separate from the brush wire 406, is attached to the front of the brush 400. The receiver bodies 402a-c and bristles 404 may free and break apart material left within the channel 500, which is then captured by the receiver bodies 402a-c and removed with the brush 400. FIGS. 5B and 5C show that the receiver bodies 402a-c are sufficiently elastic to deform in shape within the channel 500, thus fitting within the channel while thoroughly contacting the channel walls.

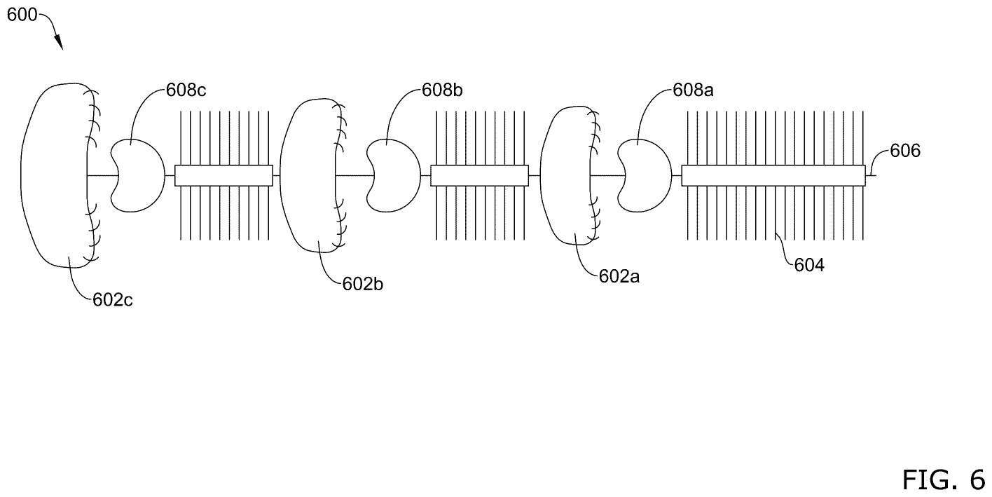

FIG. 6 shows a brush 600 which includes the components described above with respect to the brush 400, but with the addition of router bodies 608a-c in front of each of the receiving bodies 602a-c. The bristles 604 are in front of the router bodies 608a-c, and all the components are attached to the brush wire 606 as above. In some implementations, each router is sized to its matching receiver, so that the diameter of the front router 608a is the smallest and the diameter of the rear router 608c is the largest. Each router 608 is smaller in diameter than its associated receiver 602. The router body acts to divert fluid flow towards the receiver body in order to facilitate the trapping and removal of material from the endoscopic channel. The router body 608a-c may be made of the same material as the receiver bodies.

The size, shape, and number of the brush bodies may be specially selected such that, in many cases, the manual cleaning step is complete after the brush passes through the endoscopic channel a single time. The multiple sections of bristles are sufficient to remove all significant material from the inner walls, while the receiving bodies are sized to capture and carry all significant debris all the way to the end of the channel. Thus, when the correct brush is selected for the diameter, length, and material of the channel, the user performing the cleaning will find that further manual cleaning is not necessary after a single use of the brush.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A brush for cleaning an endoscopic medical device, comprising:
a wire;
a plurality of receiver bodies disposed along the wire and attached to the wire, each receiver body having a concave shape oriented in the same direction along the wire defining a front direction; and
in front of each of the plurality of receiver bodies, a plurality of bristles extending transversely from the wire and attached to the wire.

2. The brush of claim 1, wherein a rearmost body of the plurality of receiver bodies is larger than a frontmost body of the plurality of receiver bodies.

3. The brush of claim 1, wherein the plurality of receiver bodies consists of a front body, a middle body, and a rear body.

4. The brush of claim 3, wherein the front body has a diameter smaller than a diameter of the middle body, and wherein the diameter of the middle body is smaller than a diameter of the rear body.

5. The brush of claim 1, wherein each of the plurality of receiver bodies is made of the same material.

6. The brush of claim 5, wherein the material is viscoelastic.

7. The brush of claim 1, wherein the plurality of bristles are oriented radially about the wire to form a cylindrical volume defining a diameter.

8. The brush of claim 7, wherein a rearmost body of the plurality of receiver bodies has a diameter that is larger than the diameter defined by the plurality of bristles.

9. The brush of claim 7, wherein a frontmost body of the plurality of receiver bodies has a receiver that is equal to the diameter defined by the plurality of bristles.

10. The brush of claim 1, wherein the wire extends to the front of the brush a length significantly longer than a total length of the wire including the plurality of receiver bodies and the plurality of bristles.

11. The brush of claim 1, further comprising a plurality of router bodies disposed along the wire, each router body in front of a different body of the plurality of receiver bodies.

12. The brush of claim 11, wherein each of the plurality of router bodies and each of the plurality of receiver bodies are made of the same viscoelastic material.

13. A method of cleaning an endoscopic channel, comprising:
passing a brush through the endoscopic channel, the brush comprising:
a wire;
a plurality of receiver bodies disposed along the wire and attached to the wire, each receiver body having a concave shape oriented in the same direction along the wire defining a front direction; and
in front of each of the plurality of receiver bodies, a plurality of bristles extending transversely from the wire and attached to the wire; and
applying a fluid to rinse the endoscopic channel.

14. The method of claim 13, further comprising:
inspecting the endoscopic channel for debris; and
determining that the endoscopic channel is free of debris after a single pass of the brush through the endoscopic channel.

15. The method of claim 13, further comprising:
subjecting the endoscopic channel to high level disinfection.

16. The method of claim 13, wherein a rearmost body of the plurality of receiver bodies is larger than a frontmost body of the plurality of receiver bodies.

17. The method of claim 13, wherein the plurality of receiver bodies consists of a front body, a middle body, and a rear body.

18. The method of claim 17, wherein the front body has a diameter smaller than a diameter of the middle body, and wherein the diameter of the middle body is smaller than a diameter of the rear body.

19. The method of claim 13, wherein each of the plurality of receiver bodies is made of the same viscoelastic material.

20. The method of claim 13, wherein the plurality of bristles of the brush are oriented transverse from the wire and are oriented radially about the wire to form a cylindrical volume defining a diameter.
